(19) United States
(12) Patent Application Publication
(10) Pub. No.: US 2015/0359934 A1
(43) Pub. Date: Dec. 17, 2015

(54) Articles for Tissue Regeneration with Biodegradable Polymer

(71) Applicant: CorMartix Cardiovascular, Inc., Roswell, GA (US)

(72) Inventor: Robert G Matheny, Norcross, GA (US)

(21) Appl. No.: 14/832,226

(22) Filed: Aug. 21, 2015

Related U.S. Application Data
(63) Continuation-in-part of application No. 14/682,188, filed on Apr. 9, 2015.
Application No. 14/682,188 is a continuation of application No. 11/747,028, filed on May 10, 2007, now Pat. No. 9,034,367.

(51) Int. Cl. A61L 27/36; A61L 27/58; A61L 27/18; A61L 27/16; A61L 27/54; A61L 27/50

(52) U.S. Cl. CPC A61L 27/3633; A61L 27/3629; A61L 27/54; A61L 27/50; A61L 27/18; A61L 27/16; A61L 27/58; A61L 27/3641; A61L 2300/414; A61L 2430/34

(57) ABSTRACT

The invention is also directed to particulate articles comprising extracellular matrix components that are encased in a biodegradable polymer composition. Methods of for treating damaged tissue and regenerating new tissue at sites of the damaged tissue by placing a particulate article in mammals are claimed.

### CROSS-REFERENCES TO RELATED APPLICATIONS

This application is a continuation-in-part of U.S. application Ser. No. 14/682,188, which is a continuation of U.S. application Ser. No. 11/747,028, filed on May 10, 2007, now U.S. Pat. No. 9,034,367.

### FIELD OF THE INVENTION

The invention relates to articles made with extracellular matrix materials.

### BACKGROUND OF THE INVENTION

Tissue regeneration has been accomplished by using extracellular matrix material derived from mammalian tissues. Some of these mammalian tissues that have been described in patent literature include small intestine submucosa (SIS), liver basement membrane (LBM), urinary bladder submucosa (UBS) and stomach submucosa (SS). See U.S. Pat. No. 5,554,389, U.S. Pat. No. 4,902,508, and U.S. Pat. No. 5,281,422. Enamel matrices, which are the extracellular matrix around forming teeth, are described in U.S. Pat. No. 7,033,611. Extracellular matrices from these tissues have been isolated and described as solid materials (sheets and particulates), and in fluidized or emulsion forms made by reconstituting particulate in a suitable buffer. Presently, these extracellular matrix compositions are used for tissue grafting, wound healing, and tissue regeneration purposes.

It would be advantageous to the field of tissue engineering to invent articles and compositions for effecting improved tissue regeneration.

### SUMMARY OF THE INVENTION

The invention is an article comprising a sheet of extracellular matrix encasing a composition comprising a biodegradable polymer.

The invention is also an article comprising a conduit formed of an outer tube and a concentric inner tube, each tube comprising extracellular matrix sheets, said conduit having a space between said outer tube and said inner tube, said space occupied with a composition comprising a biodegradable polymer.

The invention is also a particulate article comprising a particulate extracellular matrix component that is encased in a biodegradable polymer composition.

The invention is further a method comprising identifying a defect or wound in tissue in a mammal, providing an article comprising a sheet of extracellular matrix encasing a composition comprising a biodegradable polymer composition, or a particulate article comprising a particulate extracellular matrix component that is encased in a biodegradable polymer composition, contacting said defect or wound with said article, and regenerating tissue at said defect or healing said wound, whereby said sheet or particulate article biodegrades in said mammal at a designed rate.

These and other elements of the invention are detailed below.

### BRIEF DESCRIPTION OF THE DRAWINGS

FIG. 1A depicts an article having two sheets of extracellular matrix encasing a composition comprising biodegradable polymer;

FIG. 1B depicts an article having two sheets of extracellular matrix encasing a composition comprising a biodegradable polymer having the ends of the article closed to fully encase the composition;

FIG. 1C depicts concentric tubes forming a conduit that encases a biodegradable polymer placed between the two concentric tubes that form the conduit; and

FIG. 2 depicts a particulate article comprising a particulate extracellular matrix component that is encased in a biodegradable polymer composition.

### DETAILED DESCRIPTION OF THE INVENTION

The invention is an article made of one or more extracellular matrix sheets encasing a composition comprising a biodegradable polymer. The article can comprise two or more sheets of extracellular matrix. Two sheets can sandwich an amount of the composition comprising a biodegradable polymer.

The invention is also an article comprising concentric tubes of extracellular matrix forming a conduit with a space between the tubes that is filled with a biodegradable polymer composition.

The article of concentric tubes is useful for tissue regeneration or wound healing at a vessel in the body. The vessel can be for example a blood vessel, such as an artery or vein. The vessel can also be a large intestine, or small intestine. The vessel can also be any number of tubes that connect to and from the organs in the body, for example, in the reproductive organs (e.g. fallopian tubes, prostate), bladder, urinary tract, gastrointestinal tract, lung, heart and kidney vessels, and any tubular part of the mammalian body in need of repair. The sizes and design of the article will be adjusted for the location that the article is being placed in the body, thus, for example, a large intestine will receive a large conduit, and an artery or vein a much smaller one.

The term “particulate” is a term that is meant to include an extracellular matrix component having a size suitable for forming an emulsion.

The inventions are useful for placing in a mammal in need of tissue regeneration to effect tissue regeneration at the site of placement of the article or device. The extracellular matrices used in the articles (i.e. the sheets of extracellular matrix) can be from one or more than one source of extracellular matrix in a mammal. For example, one sheet article can be SIS and one sheet article can be SS, or both sheet articles (or tubes) can be SIS.

The biodegradable polymer composition can change consistency in response to a physiological condition. Thus, the biodegradable polymer composition outside the body can be a certain consistency (for example a liquid) and then when placed in the body the consistency can change in response to the change in pH, temperature, or enzymatic activity present in the body at the site of placement of the article.

Accordingly, and optimally, the biodegradable polymer composition might be a liquid before placement in the body and gel upon contact with the environment in the body with the physiological condition that makes the biodegradable polymer composition gel (e.g. pH difference, temperature difference, or enzymatic activity).

The biodegradable polymer composition can comprise an additional element, for example a protein, a cell, or a drug.

The article that is a conduit can be formed of an outer tube and a concentric inner tube. Each tube can comprise extracellular matrix sheets, and the conduit can have a space between the outer tube and the inner tube. The space is occupied with a composition comprising a biodegradable polymer.

As with the other articles of the invention, the biodegradable polymer composition between the two pieces of extracellular matrix can also comprise an additional component, such as a protein, a cell, or a drug.

As indicated above, the invention is also a particulate article comprising a particulate extracellular matrix component that is encased in a biodegradable polymer composition.

The biodegradable polymer composition can also change consistency in response to a physiological condition, as described earlier.

The biodegradable polymer composition in all these articles can be designed to degrade at a predetermined or designed rate. For example, if it is determined that the extracellular matrix sheets that encase the biodegradable polymer composition need about 3 months of support before they have more or less fully assimilated as new tissue at the site, then the biodegradable polymer composition should be designed to be fully degraded by 3 months of presence in the body. The biodegradable polymer composition can be adjusted to degrade at different rates depending on the biodegradable polymer composition and its known rate of degradation in the body, as well as by manipulating other factors such as the properties of the biodegradable polymer composition and specific composition of the biodegradable polymer composition, the quantity of biodegradable polymer composition placed in the article, as well as by manipulating other parameters in the biodegradable polymer composition.

The invention is also a method of using the articles of the invention. The articles can be used to generate new tissue at a site of defect, or to heal a wound in the tissue. Most commonly humans are the subjects for this treatment, but any mammal can be treated using these methods. Mammals such as horses, dogs, cats and other mammals (particularly domesticated mammals) in need of tissue regeneration can be treated with these methods.

The method includes that a defect or wound in mammalian tissue is identified in a subject mammal. The defect or wound in the subject mammal is treated by contacting the defect or wound with an article of the invention. The articles can be a flat sandwich type sheet configuration, encasing the biodegradable polymer composition, or a conduit of two concentric tubes of extracellular matrix that encase the biodegradable polymer composition between the concentric tubes, or a particulate article comprising a particulate extracellular matrix component that is encased in a biodegradable polymer composition. As a result of placing the article at the site in the mammal, tissue is regenerated at the defect or the wound is healed, about which time the biodegradable polymer in the composition has degraded or nearly degraded.

The tissue to be repaired using the articles of the invention, and the methods of the invention include myocardial tissue, pancreatic tissue, and liver tissue, for example.

As with the articles of the invention, the method invention provides that the extracellular matrix and/or biodegradable polymer composition can also include an additional component, such as a protein, cell or drug.

The articles of the invention are made up of a composition comprising a biodegradable polymer. The biodegradable polymer composition provides support for the extracellular matrix components of the articles and then slowly degrades as the matrix integrates into the surrounding tissue and becomes new tissue in the animal.

Biodegradable polymers can be either natural or synthetic. A material is called degradable with respect to specific environmental conditions if it undergoes a degradation to a specific extent within a given time measured by specific standard test methods. Degradation is also an irreversible process leading to a significant change of structure of a material, typically characterized by a loss of properties (e.g. integrity, molecular weight, structure or mechanical strength) and/or fragmentation. Degradation is affected by environmental conditions and proceeds over a period of time comprising one or more steps. Disintegration is the falling apart into very small fragments of packaging or packaging material caused by degradation mechanism. The general criteria for selecting a biodegradable polymer for use as a in a biodegradable polymer composition is to match the mechanical properties and the time of degradation to the needs of the application. The ideal biodegradable polymer composition for a particular application would be configured so that it has mechanical properties that match the application, remaining sufficiently strong until the surrounding tissue has healed; does not invoke an inflammatory or toxic response; is metabolized in the body after fulfilling its purpose, leaving no trace; is easily processable into the final product form; demonstrates acceptable shelf life and is easily sterilized. Some biodegradable polymers suitable for use in the biodegradable polymer compositions and, hence, articles and devices of the invention include (but are not limited to) the following: polyglycolide (PGA), polylactide (PLA), poly-epsilon-caprolactone, poly dioxanone (a polyether-ester), poly lactide-co-glycolide, polyamide esters, polyalkalene esters, polyvinyl esters, polyvinyl alcohol, polyanhydrides, and in addition to other biodegradable polymers under investigation for similar applications. Natural biodegradable polymers that might be used include (but are not limited to) polysaccharides (e.g. starch and cellulose), proteins (e.g. gelatin, casein, silk, wool), polyesters (e.g. polyhydroxyalkanoates), and others (e.g. lignans, shellac, natural rubber). This list is not intended to be exhaustive of all the biodegradable polymers suitable for use in the compositions.

The encasing of the biodegradable polymer composition can be accomplished by laminating the ends of the sheet articles to enclose the biodegradable polymer composition inside. The biodegradable polymer composition can be sandwiched between two sheets of extracellular matrix with the edges of the sheets laminated together, or somehow made to close either fully or partially to encase the biodegradable polymer composition inside.

The article can also be made by providing a biodegradable polymer composition that is enclosed by a single sheet of extracellular matrix that folds over on itself to hold the biodegradable polymer composition. The three sides of the sheet can be laminated to itself to form an article that encases the biodegradable polymer composition and which is folded on one edge.

The sheet articles can be from the same source of extracellular matrix, i.e. both or all sheets can be made of SIS from a pig. The sheet articles can also be from different sources of extracellular matrix, for example the first sheet article is SIS, and the second sheet article is SS. Both the SIS and SS can be from the same species of mammal (pig) or each from a different species of mammal (SIS from pig, and SS from cow). Accordingly, such extracellular matrices as LBM and UBS can be used for making the sheet articles, and mixed and matched according to the needs of the animal being treated. For example, it may be beneficial to have an underside sheet of a higher tensile strength material such as SIS, and a topside sheet of a weaker strength such as LBM. Likewise with the concentric tubes the outer tube can be of SIS and the inner tube can be of LBM or UBS, the inner tubes providing less tensile strength but equal or greater tissue regenerative potential than the outer tube of SIS.

The sheet articles can be laminated to each other at the edges around an amount of composition comprising the biodegradable polymer that then becomes encased in the two sheet articles upon lamination of the outer sheet articles to each other. The lamination of the two outer sheet articles together can be partial or complete, so that the biodegradable polymer composition inside can be entirely contained within the two sheet articles, or can be permitted to ooze out from between the sheet articles upon placement in the subject receiving treatment.

The two sheet articles may also be attached to each other by quilting of the sheet articles in the middle of the sheet articles much like a quilt is assembled when made of 2 or more layers of fabric.

The particulate extracellular matrix components of the particulate articles can be formed by various conventional means. By way of example, the particulate extracellular matrix components of the particulate articles can be dip coated in a biodegradable polymer composition.

The biodegradable polymer composition can be a mixture of more than one biodegradable polymer. Accordingly, in between the sheets of extracellular matrix can be of a mixed source of biodegradable polymers, so that for example a biodegradable polymer composition can be a 50:50 mixture of PGA and PGLA, or some other permutations of mixtures that will serve the purpose of the biodegradable polymer composition in the article.

The biodegradable polymer composition can also be mixed with a certain percentage of liquid, gel or particulate extracellular matrix. The function of the biodegradable polymer composition will be to support the extracellular matrix sheets or tubes until they have assimilated into new tissue in the animal, and thus the strength of the biodegradable polymer composition and the rate of its degradation in the animal will be two of the important parameters to adjust as the biodegradable polymer composition is fine-tuned for a particular specific application in a particular subject being treated.

Mammalian tissue sources are any tissue having an extracellular matrix that can be isolated from a mammal and decellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ.

The mammal from which the extracellular matrix sheets are derived can be any mammal, including (but not limited to): humans, cows, pigs, dogs, cats, horses, rodents or any other mammal who provides the necessary material. Any mammal can potentially contribute extracellular matrix for making the sheet articles or extracellular matrix components of the invention.

The forms of the extracellular matrices that make up the articles can be in the form of sheet articles that can be folded or manipulated to both encase the biodegradable polymer composition, and form the desired shape for the article or device. Thus, the sheet articles can form sandwiches, or conduits, or other shapes such a triangular shapes, circular shapes, irregular shapes, and shapes tailored or designed to fit specific locations in the animal.

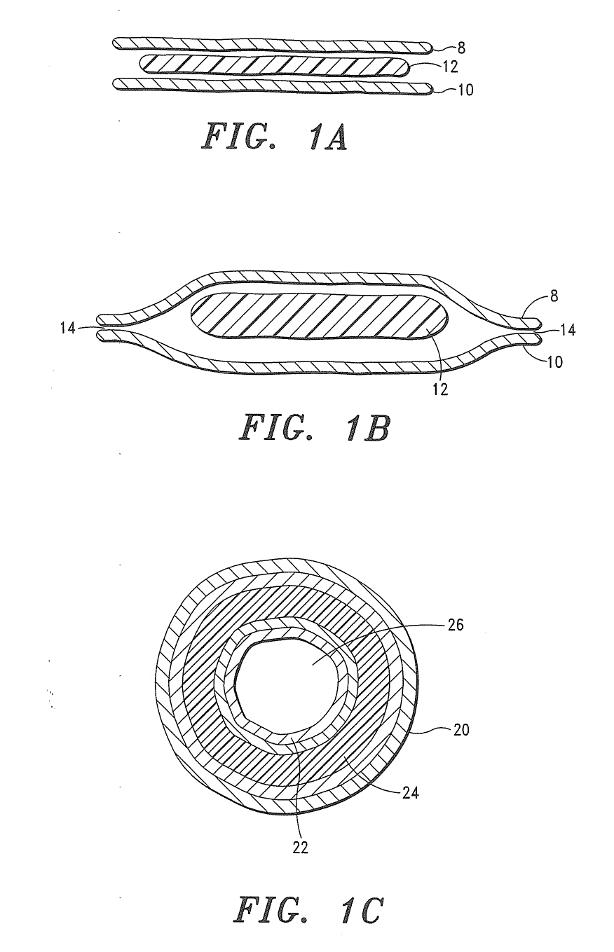

The sheets articles can have any number of shapes, e.g. square, rectangular, triangular, circular, or an irregular shape. The shape of the article can be tailored to fit the site where the article will be introduced into the body. Accordingly, the biodegradable polymer compositions that make up the center or encased portion of the article can be any of these forms, encased in one or more sheets of extracellular matrix. The form can also be a conduit having two concentric tubes with the space between the tubes filled with the biodegradable polymer composition. (see FIG. 1C)

The particulate extracellular matrix compositions of the particulate articles can be formed by various conventional means. In some embodiments, the extracellular matrix is formed into a sheet, fluidized (or hydrated), if necessary, frozen and ground.

In some embodiments of the invention, the ground extracellular matrix is subsequently filtered to achieve a desired particulate size. Thus, in some embodiments, the extracellular matrix has a particulate size no greater than 2000 microns. In some embodiments, the extracellular matrix preferably has a particulate size no greater than 500 microns. In a preferred embodiment, the extracellular matrix has a particulate size in the range of about 20 microns to about 300 microns.

The biodegradable polymer composition is useful to the articles of the invention because it will hold a shape and retain a presence for a temporary period of time. As the sheet of extracellular matrix forms new tissue, the biodegradable polymer composition will help the sheet articles maintain a position or shape necessary until the tissue is formed. Ideally the degradation of the biodegradable polymer composition is designed to match the needs of the application so that the biodegradable polymer composition degrades as the new tissue is being formed, and eventually is completely gone by the time the new tissue is strong enough to fully support its new application in the body.

Extracellular matrix sheets and particulate extracellular matrix components and any incidental emulsion can be obtained from the tissues of mammals by processes such as described in U.S. Pat. No. 5,554,389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Matrices can be used in whole or in part, so that for example, an extracellular matrix can contain just the basement membrane (or transitional epithelial layer) with the sub-adjacent tunica propria, the tunica submucosa, tunica muscularis, and tunica serosa. The extracellular matrix composition can contain any or all of these layers, and thus could conceivably contain only the basement membrane portion, excluding the submucosa. However, since the submucosa is thought to contain and support the active growth factors and other proteins necessary for in vivo tissue regeneration, the extracellular matrix composition from any given source will contain the active extracellular matrix portions that support cell development and differentiation and tissue regeneration once placed in a live mammal. Thus, it is generally understood by persons of skill in the art that the extracellular matrix of any of the mammalian tissue consists of several basically inseparable layers broadly termed extracellular matrix. Where layers can be separated these separate layers can electively be included in the extracellular matrix composition, depending on whether they serve the purpose that is the goal of the article.

The extracellular matrix can be made into a particulate to form the particulate extracellular matrix compositions of the invention and fluidized as described in U.S. Pat. No. 5,275,826 to Badylak, U.S. Pat. No. 6,579,538 to Spievack, and U.S. Pat. No. 6,933,326 to Griffey. Fluidized or emulsified compositions (the liquid or semi-solid forms) can be present at a certain concentration, for example at a concentration of extracellular matrix greater than about 0.001 mg/ml. The concentration of these liquid or semi-solid components of the extracellular matrix composition can be in a range from about 0.001 mg/ml to about 200 mg/ml. The concentrations can further be found in more specific ranges such as for example the following set of ranges: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml, and about 40 mg/ml. to about 42 mg/ml. This set of ranges is exemplary and not intended to be exhaustive. It is contemplated that any value within any of these specifically listed ranges is a reasonable and useful value for a concentration of a liquid or semi-solid component of the composition.

Turning now to the figures, FIG. 1A depicts a sandwich configuration of the article. Element 10 is a bottom sheet of extracellular matrix. Element 8 is a top sheet of extracellular matrix. Element 12 is the composition of biodegradable polymer sandwiched between sheet 8 and 10.

FIG. 1B depicts the sandwich with closed ends, the article encasing the composition of biodegradable polymer. Bottom sheet 10 and top sheet 8 are closed or nearly closed at points 14 to encase the biodegradable polymer composition 12.

FIG. 1C depicts concentric tubes having a biodegradable polymer composition in between them. Outer concentric tube 20 exists on the outside of the article. The composition having biodegradable polymer 24 is between outer tube 20 and inner concentric tube 22. Space 26 exists in the center of the concentric tubes through which fluid or other material can pass after the article is placed in the body at the site where it is needed.

The composition comprising a biodegradable polymer can further include one or more additional components to aid in some aspect of the tissue regenerative process. The additional component will generally be part of the composition comprising biodegradable polymer that is placed between the sheets of matrix. Thus, the additional component can help to regenerate tissue, heal a wound, better recruit stem cells, manipulate the immune environment in a beneficial way, therapeutically treat the local environment, or otherwise contribute to some aspect of the process for which the composition is being used.

Thus, the additional component can be a cell, a protein or a drug (e.g. a small molecule). The cell can be a stem cell, such as, for example a of human embryonic stem cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, an embryonic stem cell, a parenchymal cell, an epithelial cell, an endothelial cell, a mesothelial cell, a fibroblast, a myofibroblast, an osteoblast, a chondrocyte, an exogenous cell, an endogenous cell, a stem cell, a hematopoetic stem cell, a pluripotent stem cell, a bone marrow-derived progenitor cell, a progenitor cell, a myocardial cell, a skeletal cell, a fetal cell, an embryonic cell, an undifferentiated cell, a multi-potent progenitor cell, a unipotent progenitor cell, a monocyte, a cardiomyocyte, a cardiac myoblast, a skeletal myoblast, a macrophage, a capillary endothelial cell, a xenogenic cell, an allogenic cell, an adult stem cell, and a post-natal stem cell. This list is not intended to be exhaustive.

The protein can be for example a growth factor, or any other type or protein that might stimulate some part of the tissue regenerative process a collagen, a proteoglycan, a glycosaminoglycan (GAG) chain, a glycoprotein, a growth factor, a cytokine, a cell-surface associated protein, a cell adhesion molecule (CAM), an angiogenic growth factor, an endothelial ligand, a matrikine, a matrix metalloprotease, a cadherin, an immunoglobin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor alpha (TGF-alpha), transforming growth factor beta (TGF-beta), fibroblast growth factor-2 (FGF-2) (also called basic fibroblast growth factor (bFGF)), thrombospondin, osteopontin, angiotensin converting enzyme (ACE), and vascular epithelial growth factor (VEGF). This list is not intended to be exhaustive.

The additional component can also be a drug, such as an agent that has therapeutic properties. The drug can be bioactive and play some role in the process of tissue regeneration, for example, or act as an antibiotic, antiviral, or other active therapeutic agent serving a purpose in the composition as a whole, also by example. The drug can be a small molecule, or any other agent having therapeutic properties. The drug can have the capacity to treat the patient locally at the site of placement of the article, as for example a local antibiotic or anti-inflammatory agent. The drug may also alternatively have the capacity to treat the patient or subject systemically, as with a molecule that can travel in the blood stream from the site of placement of the article to other parts of the body where it can have effects, e.g. a therapeutic agent that can have effects in another system in the patient.

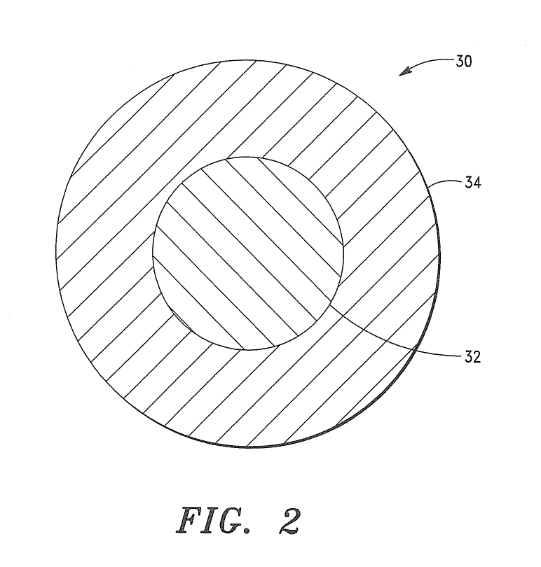

FIG. 2 depicts one embodiment of a particulate article of the invention. As illustrated in FIG. 2, the article 30 comprises a base particulate component 32 that is encased in a biodegradable polymer component 34.

As indicated above, the base particulate component 32 can comprise an extracellular matrix composition.

In a preferred embodiment of the invention, a plurality of the particulate articles shown in FIG. 2, are employed to form a biomaterial composition for regenerating tissue and/or healing a wound in damaged mammalian tissue.

Thus, in some embodiments the biomaterial composition comprises a plurality of particulate components comprising an extracellular matrix composition, the extracellular matrix composition comprising at least one extracellular matrix material, each of the plurality of particulate extracellular matrix components being encased in a biodegradable polymer composition, the particulate components being configured to induce tissue regeneration and/or wound healing when delivered to damaged mammalian tissue.

The extracellular matrix compositions and/or biodegradable polymer composition can also include any of the aforementioned cells, proteins and/or drugs.

The invention contemplates using the articles for regenerating tissue as a defect or healing a wound in mammalian tissue. The defect can be a cut, disease, wound, burn, scar, necrosis, or other abnormality that would be beneficial to the patient to treat. Regenerating tissue at the defect can be one response elicited from the step of placing the extracellular matrix sheets in contact with the defect, while the biodegradable polymer composition can help hold a shape or support the extracellular matrix sheet until the tissue regenerates or the wound heals. If the defect is a wound in need of healing, wound healing may be another response that occurs as a result of placing the article and, hence, extracellular matrix at the wound site. Any term that identifies that the tissue could benefit from healing or where the concept of tissue regeneration fits within the scope of the use for the extracellular matrix composition can be used to describe the process that is the goal of placing the article in the patient.

Therapeutically effective amount is a term meant to capture the idea that you need to apply enough of an element of the composition in sufficient strength so that the composition can have a positive effect on the tissue that is being treated in the subject. Thus, the term therapeutically effective amount applies to the additional components added to the composition comprising the biodegradable polymer. The term therapeutically effective amount may therefore apply to a quantity of matrix, or a size of a sheet of matrix, or a volume or weight of powder, or a concentration of liquid or emulsion. That the amount is therapeutically effective is determined by the composition's ability to have a regenerative or wound healing effect at the site where the composition contacts the tissue. A therapeutically effective amount is determinable by routine testing in patients with wounds or defects. A minimally therapeutically effective amount would be considered sufficient composition to contact amply all of the wound or defect in the tissue.

Regenerating tissue is the ability to make tissue regrow, an organ regrow itself, and for new tissue to reform without scarring. Healing a wound is the ability of the tissue to heal without scarring, or with less scarring than would have occurred without the extracellular matrix component or article formed therefrom.

All references cited are incorporated in their entirety. Although the foregoing invention has been described in detail for purposes of clarity of understanding, it will be obvious that certain modifications may be practiced within the scope of the appended claims.

What is claimed is:

1. A biomaterial composition for treating damaged biological tissue, comprising:
a plurality of particulate articles comprising a particulate extracellular matrix component comprising an extracellular matrix composition, said extracellular matrix composition comprising at least one decellularized extracellular matrix material, each of said plurality of particulate extracellular matrix components being encased in a biodegradable polymer composition comprising a biodegradable polymer, said biomaterial composition being configured to induce tissue regeneration and healing when delivered to wounded tissue.

2. The biomaterial composition of claim 1, wherein said extracellular matrix material comprises mammalian tissue selected from the group consisting of small intestine submucosa (SIS), stomach submucosa (SS), liver basement membrane (LBM) and urinary bladder submucosa (UBS).

3. The biomaterial composition of claim 1, wherein said biodegradable polymer is selected from the group consisting of polyglycolide (PGA), polylactide (PLA), poly ε-caprolactone, poly dioxanone, polylactide-co-glycolide, polyamide esters, polyalkalene esters, polyvinyl esters, polyvinyl alcohol, and polyanhydrides.

4. The biomaterial composition of claim 1, wherein said particulate articles further comprise a growth factor selected from the group consisting of transforming growth factor alpha (TGF-α), transforming growth factor beta (TGF-β), fibroblast growth factor-2 (FGF-2) and vascular epithelial growth factor (VEGF).

5. A method for treating damaged biological tissue, comprising:
identifying damaged tissue in a mammal;
providing a biomaterial composition comprising a plurality of particulate articles comprising a particulate extracellular matrix component, said particulate extracellular matrix component comprising an extracellular matrix composition comprising at least one decellularized extracellular matrix material, each of said plurality of particulate extracellular matrix components being encased in a biodegradable polymer composition comprising a biodegradable polymer;
administering said biomaterial composition to said damaged tissue, wherein said biomaterial composition induces regeneration and healing of said wounded tissue.

6. The method of claim 1, wherein said extracellular matrix material comprises mammalian tissue selected from the group consisting of small intestine submucosa (SIS), stomach submucosa (SS), liver basement membrane (LBM) and urinary bladder submucosa (UBS).

7. The method of claim 1, wherein said biodegradable polymer is selected from the group consisting of polyglycolide (PGA), polylactide (PLA), poly ε-caprolactone, poly dioxanone, polylactide-co-glycolide, polyamide esters, polyalkalene esters, polyvinyl esters, polyvinyl alcohol, and polyanhydrides.

8. The method of claim 1, wherein said particulate articles further comprise a growth factor selected from the group consisting of transforming growth factor alpha (TGF-α), transforming growth factor beta (TGF-β), fibroblast growth factor-2 (FGF-2) and vascular epithelial growth factor (VEGF).
